(19) United States
(12) Patent Application Publication
(10) Pub. No.: US 2020/0060959 A1
(43) Pub. Date: Feb. 27, 2020

(54) METHOD FOR PREPARING AN AQUEOUS HYALURONIC ACID GEL

(71) Applicant: BIOXIS PHARMACEUTICALS, LYON (FR)

(72) Inventors: Frédéric BERTAINA, POULX (FR); Alexandre GUERRY, LES ABRETS (FR); Caroline CECCALDI, SAINT ANTONIN NOBLE VAL (FR)

(73) Assignee: BIOXIS PHARMACEUTICALS, LYON (FR)

(21) Appl. No.: 16/609,283

(22) PCT Filed: May 17, 2018
(86) PCT No.: PCT/EP2018/062871

§ 371 Date: Oct. 29, 2019

(30) Foreign Application Priority Data
May 18, 2017 (FR) 1754397

(51) Int. Cl. A61K 8/73; A61K 8/04; A61Q 19/08; A61L 27/52; A61L 27/20; C08B 37/08

(52) U.S. Cl. CPC A61K 8/735; A61K 8/042; A61Q 19/08; A61L 27/52; A61L 2400/06; C08B 37/0072; A61K 2800/805; A61K 2800/91; A61L 27/20

(57) ABSTRACT

A method for preparing an aqueous hyaluronic acid gel, including the following steps: a) preparing a crosslinked aqueous hyaluronic acid gel, b) homogenising the aqueous gel formed in step (a) by rolling, c) neutralising the aqueous gel homogenised in step (b). Also, an aqueous hyaluronic acid gel able to be obtained by such a method. Further, the cosmetic use of such an aqueous gel in tissue repair or reconstruction, in particular for filling lines and wrinkles, or the medical use of the aqueous gel in tissue repair or reconstruction.

The present invention relates to a method for preparing a homogeneous aqueous hyaluronic acid gel, to the aqueous gel thus obtained and to the uses thereof, in particular for filling wrinkles and fine lines.

Collagen has long been the filling product of choice for the face, in particular for filling wrinkles and fine lines or else for redefining the lips. However, since the marketing of hyaluronic acids, the latter are increasingly used. Indeed, added to the biodegradability of collagen, which is considered to be too fast, are the problems of safety linked to the animal (bovine or porcine) origin thereof.

The injection of hyaluronic acid has two advantages: an immediate mechanical filling effect and the absence of inflammatory phenomena owing to its biocompatibility. When it is administered in a linear (non-crosslinked) form, hyaluronic acid has excellent biocompatibility but is rapidly degraded by the body (in around one week). The lifetime of injected products based on hyaluronic acid has been able to be significantly prolonged up to around 12 months by the use of crosslinked hyaluronic acid. Indeed, crosslinked hyaluronic acid is in the form of a cohesive gel having viscoelastic properties that are particularly advantageous for wrinkle-filling products.

However, during the crosslinking, particles of “hard zones” form within the hyaluronic acid gel rendering it inhomogeneous. These hard zones affect the injectability of the product and are liable to pose problems of tolerance for the patient. It is therefore essential to eliminate these hard zones from crosslinked hyaluronic acid gels in order to have a perfectly homogeneous product for the administration thereof. Conventionally, crosslinked hyaluronic acid gels are homogenized by screening or by extrusion. These methods only permit a partial elimination of the hard zones. Furthermore, the shear stress exerted on the gels leads to a deterioration of its structure and of its viscoelastic properties. The gel subjected to a screening, a filtration or an extrusion is not therefore perfectly homogeneous and it experiences a reduction in viscosity. Once injected, it risks migrating into the tissues and degrading more rapidly. Its filling properties are therefore degraded.

It is therefore desirable to have a method that makes it possible to effectively eliminate the hard zones present in crosslinked hyaluronic acid gels so as to obtain a homogeneous gel, without deteriorating its viscoelastic properties.

The present invention therefore proposes a method for preparing a homogeneous crosslinked hyaluronic acid gel, wherein the homogenization of the gel is obtained by rolling.

In particular, one subject of the present invention is, according to a first aspect, a method for preparing an aqueous hyaluronic acid gel comprising the following steps:
a) preparing an aqueous crosslinked hyaluronic acid gel,
b) homogenizing the aqueous gel formed in step (a) by rolling,
c) neutralizing the aqueous gel homogenized in step (b).

Another subject of the invention is, according to a second aspect, an aqueous hyaluronic acid gel capable of being obtained by such a method.

A further subject of the invention is, according to a third aspect, the cosmetic use of such an aqueous gel in the repair or reconstruction of tissues, in particular for filling wrinkles and fine lines, or said aqueous gel for the medical use thereof for the repair or reconstruction of tissues.

### FIGURES

FIG. 1 illustrates a rolling of the gel carried out between two rollers rotating at the same tangential speed.

FIG. 2 illustrates a rolling of the gel carried out between three rollers, the tangential speed of which increases to enable the entrainment of the gel onto the surface of the adjacent roller.

FIGS. 3, 4 and 5 illustrate the ejection forces of compositions 3, 4 and 5 of aqueous gels prepared in the exemplary embodiments.

### AQUEOUS GEL

The present invention proposes a new method for preparing aqueous hyaluronic acid gels.

A “gel” is understood, for the purposes of the present application, to mean a cohesive composition, which does not flow under its own weight, and which has viscoelastic properties that give it a certain deformability. The gel, if it is sheared, does not reform, unlike viscous fluids.

The hyaluronic acid gels, according to the invention, therefore differ from hyaluronic acid solutions. The gel/solution distinction may be observed by a rheological study under strain and a constant frequency at 25° C. in order to determine the viscous modulus G″ and the elastic modulus G′ in the linear viscoelasticity zone. Specifically, a gel within the meaning of the present invention is characterized in particular by the fact that its elastic modulus G′ is greater than the viscous modulus G″ according to the definition by Winter and Chambon (1986). In the case of a viscous solution on the other hand, the viscous modulus G″ is greater than its elastic modulus G′.

The measurements are carried out on a Discovery HR1 (TA Industries) rheometer and a 40 mm plate/plate geometry according to a continuous mode (10% strain, frequency of 1 Hz, at 25° C., for 120 s). The samples consisting of around 1.2 ml are deposited in a gap of 1000 μm.

The hyaluronic acid gels according to the invention are preferably homogeneous. A “homogeneous hyaluronic acid gel” is understood for the purposes of the present invention to mean that the crosslinked hyaluronic acid is dispersed uniformly within the gel.

The homogeneity of the hyaluronic acid gel may in particular be characterized by measuring the variation in the force for ejecting the gel through a syringe, the needle of which has an internal diameter of 300 μm (27 G TSK UTW). The measurement of the ejection force (or extrusion force) is carried out by means of a Shimadzu EZ-Test SX force bench equipped with a 50 N cell. The extrusion is carried out at 10 mm·min⁻¹ and the sampling is set at 100 point·s⁻¹. The tests are carried out with long 1 ml BD syringes equipped with a ½″ TSK 27 G needle. The acquisition is processed between the 20^{th} and 140^{th} second of extrusion in order not to take into account contacting stresses at the start and end of extrusion. At the end of the acquisition, the series of points N=f(t) (extrusion force as a function of time) is linearized. A margin of ±10% with respect to the linearity is depicted. Each intersection of the curve N=f(t) with the linearized N=f(t) straight lines N_{+10%}=f(t) and N_{-10%}=f(t) corresponds to the extrusion of a heterogeneous portion.

Thus, according to a preferred embodiment, the homogeneous hyaluronic acid gel does not have a variation of the extrusion force of more than ±10% relative to the linearized extrusion force.

### Hyaluronic Acid

The aqueous gels according to the invention comprise at least one hyaluronic acid.

Hyaluronic acid is a linear glycosaminoglycan (GAG) composed of D-glucuronic acid and N-acetyl-D-glucosamine repeat units bonded together by alternate beta-1,4 and beta-1,3 glycosidic bonds.

Hyaluronic acid has the following structure:

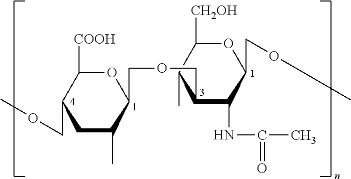

Preferably, the hyaluronic acid used in the preparation of the aqueous gel according to the invention has a molar mass of between 1 000 000 Da and 5 000 000 Da, preferably between 1 500 000 Da and 3 500 000 Da. The molecular weight may in particular be determined by Waters GPCV Alliance 2000 size exclusion chromatography, eluent: 0.1M NaNO₃ in water coupled in-line with three Wyatt detectors: a refractometer, a viscometer and a measurement of the light scattering.

The hyaluronic acid is present in the aqueous hyaluronic acid gel obtained in step a) at a content of between 1 mg/ml and 300 mg/ml, preferably between 75 and 200 mg/ml, more preferentially between 100 and 175 mg/ml.

### Aqueous Phase

Besides the hyaluronic acid, the aqueous gels according to the invention also comprise an aqueous phase.

The gel may comprise water at a content ranging from 60% to 99% by weight, relative to the total weight of the composition, preferably ranging from 70% to 99% by weight, and preferentially ranging from 80% to 99% by weight.

The gel may further comprise a polyol that is miscible with water at ambient temperature (25° C.) in particular chosen from polyols having notably from 2 to 20 carbon atoms, preferably having from 2 to 10 carbon atoms, and preferentially having from 2 to 6 carbon atoms) such as glycerol, propylene glycol, butylene glycol, pentylene glycol, hexylene glycol, dipropylene glycol, diethylene glycol; glycol ethers (having notably from 3 to 16 carbon atoms) such as mono-, di- or tripropylene glycol (C1-C4)alkyl ethers, mono-, di- or triethylene glycol (C1-C4)alkyl ethers; and mixtures thereof.

The water-miscible polyol may be present in the gel according to the invention at a content ranging from 0.1% to 20% by weight, relative to the total weight of the composition, and preferably ranging from 3% to 15% by weight.

### Preparation of an Aqueous Crosslinked Hyaluronic Acid Gel

The method according to the invention implements a first step a) of preparing an aqueous crosslinked hyaluronic acid gel.

This step a) preferably comprises at least the crosslinking, in an acid or basic medium, of said hyaluronic acid in the presence of at least one crosslinking agent.

According to a particular embodiment, the crosslinking is carried out in a basic medium and comprises at least the following steps:
dissolving at least one hyaluronic acid and/or one of the salts thereof in a basic solution having a pH of greater than 7.5, preferably greater than or equal to 10, more preferentially between 10 and 14,
crosslinking, in basic solution, said hyaluronic acid in the presence of at least one crosslinking agent.

The crosslinking in a basic medium favors the formation of ether bonds between the hyaluronic acid and the crosslinking agent, which degrade slowly.

According to another particular embodiment, the crosslinking is carried out in an acid medium and comprises at least the following steps:
dissolving at least one hyaluronic acid and/or one of the salts thereof in an acid solution having a pH of less than 6.5, preferably less than or equal to 5, more preferentially between 4.5 and 2,
crosslinking, in acid solution, said hyaluronic acid in the presence of at least one crosslinking agent.

The crosslinking in an acid medium itself favors the formation of ester bonds between the hyaluronic acid and the crosslinking agent, which degrade more rapidly than the ether bonds.

According to a preferred embodiment, the step of crosslinking the hyaluronic acid comprises at least a crosslinking, in a basic medium, of the hyaluronic acid and a crosslinking, in an acid medium, of the hyaluronic acid, so as to control the ether and ester bonds formed, and thus the rate of degradation of the crosslinked hyaluronic acid gel thus formed.

More preferentially, the step of crosslinking the hyaluronic acid comprises a first crosslinking, in a basic medium, of the hyaluronic acid followed by a crosslinking, in an acid medium, of the hyaluronic acid.

Before dissolving, the hyaluronic acid used in the method according to the invention is typically in dry form, preferably in the form of powder or flakes.

When it is used in salt form, the hyaluronic acid may preferably be a sodium salt, a calcium salt, a zinc salt or a potassium salt of hyaluronic acid, and preferably a sodium salt.

The content of linear hyaluronic acid dissolved in the aqueous solution (corresponding to the content of hyaluronic acid during step a)) is between 50 mg/ml and 300 mg/ml, preferably between 100 and 200 mg/ml.

The crosslinking of the linear hyaluronic acid dissolved in the aqueous solution is carried out in the presence of at least one crosslinking agent.

The crosslinking agent is preferably chosen from difunctional epoxides, multifunctional epoxides, bifunctional or polyfunctional esters, divinyl sulfones, carbodiimides, formaldehyde, dialdehydes and mixtures thereof, and preferably the crosslinking agent is 1,4-butanediol diglycidyl ether (BDDE, also known under the name 1,4-diglycidyloxybutane, Tetramethylene Glycol Diglycidyl Ether and under the IUPAC name 2-[4-(oxiran-2-ylmethoxy)butoxymethyl]oxirane).

The crosslinking agent is notably introduced in an amount of between 10 mg and 250 mg per gram of linear hyaluronic acid introduced in the crosslinking step.

The crosslinking step is preferably carried out at a temperature of between 30° C. and 70° C., preferably between 45° C. and 55° C., which makes it possible to catalyze the crosslinking of the hyaluronic acid.

### Homogenization by Rolling

Within the context of the method of the invention, the aqueous crosslinked hyaluronic acid gel prepared in step a) is then homogenized by rolling in order to eliminate the hard zones (aggregates formed during the crosslinking) without degrading the mechanical and viscoelastic properties of the gel.

In particular, the rolling consists of a continuous compression between at least two counter-rotating rollers, preferably three counter-rotating rollers.

The feed roller may for example rotate at a tangential speed of between 0.1 m·s⁻¹ and 5 m·s⁻¹, preferably between 0.5 m·s⁻¹ and 3 m·s⁻¹.

When the rolling is carried out between two rollers, these preferably rotate at the same tangential speed, and the gel is introduced between the two rollers, as illustrated in FIG. 1.

When the rolling is carried out between three rollers, the tangential speed of the various rollers should increase in order to enable the entrainment of the gel onto the surface of the second roller to conduct a second rolling between the 2^{nd} and 3^{rd} rollers. By way of example, as illustrated in FIG. 2, if the first roller rotates at a tangential speed ×1, the second could rotate at this tangential speed ×2, and the third at this tangential speed ×3 to enable a double rolling of the gel.

According to a preferred embodiment, the spacing between the counter-rotating rollers (also referred to as the gap) is between 20 μm and 1 mm, preferably between 20 μm and 100 μm.

The rollers may preferably be made of stainless steel, in order to be able to be easily cleaned, and optionally provided with a microporous or ceramic coating, capable of favoring the adhesion of the gel to the surface of the rollers.

According to a preferred embodiment, step b) of homogenizing by rolling is carried out for 1 minute to 2 hours, preferably between 15 minutes and 45 minutes. These relatively short rolling times may be used insofar as, within the context of the present invention, step b) of homogenizing the aqueous gel is carried out before the neutralization step c), which leads to a swelling of said aqueous gel. Specifically, before swelling, the volume of the aqueous gel to be homogenized by rolling is significantly smaller than after swelling.

### Purification

According to a particular embodiment, and in particular in the case where the neutralization is not carried out by dialysis, the aqueous crosslinked hyaluronic acid gel may be purified before or after step c) of neutralizing the aqueous gel in order to eliminate the traces of residual crosslinking agent.

According to a preferred embodiment, the purification is preferably carried out by dialysis under the conditions described above.

The purification by dialysis, besides eliminating the residual crosslinking agent, makes it possible to further refine the pH obtained after neutralization and to control the osmolarity of the gel.

The purification may lead to a new dilution of the hyaluronic acid. The content of crosslinked hyaluronic acid present in the gel after purification is between 1 mg/ml and 60 mg/ml, preferably between 5 and 50 mg/ml.

### Neutralization

After homogenization by rolling, the aqueous hyaluronic acid gel is neutralized during a step c).

This neutralization is carried out by adjusting the pH to a pH of between 6.5 and 7.5. The neutralization may be carried out by adding an acid or a base depending on whether the crosslinking was carried out in a basic or acid medium.

The neutralization leads to the dilution of the hyaluronic acid. The content of crosslinked hyaluronic acid present in the gel after neutralization is between 10 mg/ml and 100 mg/ml, preferably between 20 and 80 mg/ml.

For example, when the crosslinking has been carried out in an acid medium, the adjustment of the pH may be carried out by adding a compound such as ammonium hydroxide, sodium hydroxide, sodium hydrogen carbonate, sodium bicarbonate, sodium carbonate or derivatives thereof or a phosphate buffer solution (PBS “Phosphate Buffer Saline”).

When the crosslinking has been carried out in a basic medium, the adjustment of the pH for the neutralization may be carried out by adding a compound such as hydrochloric acid, acetic acid, phosphoric acid and sodium dihydrogen phosphate or derivatives thereof.

Alternatively, the neutralization may be carried out by dialysis. Neutralization by dialysis enables the adjustment of the pH is carried out in a very gradual manner which makes it possible to best preserve the mechanical and viscoelastic properties of the hyaluronic acid gel formed.

Dialysis is a membrane separation process for separating molecules or ions in solution. Thus, within the context of the present application, the hyaluronic acid gel according to the invention may be dialyzed against a buffer solution having a pH equal to or close to the final pH desired for the hyaluronic acid gel (target pH), i.e. between 6.5 and 7.5, preferably between 6.75 and 7.2.

The buffer solution may, for example, be a solution of phosphate buffer saline (PBS, PBS-lactic acid), of tris(hydroxymethyl)methylamine (TRIS), of TRIS saline solution (TBS), of 4-(2-hydroxyethyl)-1-piperazineethanesulfonic acid (HEPES), of 2-{[tris(hydroxymethyl)-methyl]amino}ethanesulfonic acid (TES), of 3-(N-morpholino)propanesulfonic acid (MOPS), of piperazine-N,N′-bis(2-ethanesulfonic acid), of MES (2-(N-morpholino)ethanesulfonic acid (PIPES), and of sodium chloride (NaCl).

According to a preferred embodiment, the buffer solution is a PBS (phosphate buffer saline) solution composed of an “acid” salt NaH₂PO₄, a “basic” salt Na₂HPO₄ and NaCl.

According to a particular embodiment, the buffer is physiologically acceptable, i.e. it presents no risk of intolerance or toxicity when the hyaluronic acid gel according to the invention is injected or when it is brought into contact with the tissues.

According to a particular embodiment of the invention, the dialysis may be carried out in one or several baths against a buffer solution as described above.

According to a more preferred embodiment, the dialysis may be carried out in several successive baths against buffer solutions having different pH values that are increasingly close to the final pH desired for the hyaluronic acid solution (target pH). It is thus possible to increase the pH more gradually as a function of the number of buffer baths used.

According to a preferred embodiment, in order to simultaneously control the osmolarity of the hyaluronic acid gel, the buffer used for the dialysis may be combined with a so-called neutral salt, i.e. that does not interact with the buffer, in particular a sodium salt (NaCl) or potassium salt (KCl) at a concentration of salt to achieve the osmolarity of the tissues of between 280 mOsmol·l⁻¹ and 380 mOsmol·l⁻¹.

In particular, the buffer solution may have an osmolarity of between 250 and 350 mOsm/l, preferably between 280 and 330 mOsm/l.

Within the context of the invention, the neutralization step c) leads to a swelling of the crosslinked hyaluronic acid gel. As a general rule, the swelling specifically results in an increase in the volume of hyaluronic acid gel of between 2 and 4 times relative to the volume of the aqueous crosslinked hyaluronic acid gel obtained in step a).

Within the context of the present invention, it is important that this swelling (and therefore the neutralization) is not initiated, or is initiated very little, before the rolling step b) in order to enable the use of a sufficiently narrow gap (between 20 μm and 1 mm) so as to effectively eliminate the hard zones (aggregates formed during the crosslinking) without degrading the mechanical and viscoelastic properties of the gel.

Specifically, if the rolling is carried out at the same time (as for example described in document US2013/0217872) or after the neutralization and therefore the swelling of the gel, the spacing between the rollers of the rolling mill must be increased significantly to allow the passage of the swollen gel, which occupies a larger volume. Such a spacing of greater than 1 mm between the rollers no longer makes it possible to effectively eliminate the hard zones present in the gel in order to guarantee its excellent homogeneity and its injectability. Document US2013/0217872 in this instance does not describe injectable hyaluronic acid gels, and does not seek, by the rolling, to eliminate the hard zones present in the gel. In that document, the rolling enables a mixing of the neutralized gel for a very long period of 18 to 24 h in order to achieve a swelling equilibrium.

To enable a good injectability of the gels obtained by means of the method according to the invention, it is preferable for the gel to be free of hard zones having a diameter of greater than 1 mm, preferably greater than 20 μm.

### Linear Hyaluronic Acid

According to a particular embodiment, it is possible to add a linear hyaluronic acid after step (a) of preparing the aqueous crosslinked hyaluronic acid gel so as to reduce the viscosity of the gel and thus to adjust its mechanical properties, in particular, in order to reduce the ejection force of the gel and to facilitate the filling of the syringes.

The introduction of linear hyaluronic acid may be carried out before or after step b) of homogenizing by rolling, the neutralization (dilution) step c) or the purification step described above. According to a preferred embodiment, the introduction of linear hyaluronic acid may be carried out before or after the purification step described above.

The amount of linear hyaluronic acid introduced into the crosslinked hyaluronic acid gel is preferably less than or equal to the amount of crosslinked hyaluronic acid present in the gel after neutralization and optionally purification, so as not to further dilute the hyaluronic acid.

In particular, the content of crosslinked hyaluronic acid present in the gel after purification is between 0.1 mg/ml and 100 mg/ml, preferably between 1 and 50 mg/ml.

### Additional Polymer

According to a particular embodiment, the aqueous hyaluronic acid gel also comprises at least one additional polymer other than the hyaluronic acid, such as chondroitin, cellulose, alginate, polycaprolactone, polylactic acid, polyglycolic acid, collagen, silk, PTFE and derivatives thereof.

The additional polymer may be introduced during step a), before the crosslinking of the hyaluronic acid in order to result in a co-crosslinking of the hyaluronic acid with the additional polymer, or after step a) of preparing the aqueous crosslinked hyaluronic acid gel, and in particular before step b) of homogenizing by rolling.

The additional polymer may for example be introduced at a content ranging from 0.1% to 5%, preferably from 0.5% to 4%.

### Injectable Compositions

According to a preferred embodiment, the aqueous gel prepared according to the method of the invention is injectable.

An injectable gel is understood, for the purposes of the present invention, to mean a composition that is in gel form having satisfactory properties of injectability (or syringeability, i.e. ease of injection owing to a more or less satisfactory flow through a needle into a syringe), and in particular capable of being injected by means of a syringe having a needle with an internal diameter approximately equal to 300 μm. For the purposes of the present application, considered to be injectable from a rheological point of view are gels preferably having a viscosity of less than or equal to 10 000 Pa·s and a loss factor (Tan δ) of between 0.01 and 5.

The rheological measurements (G′, G″ and Tan δ) are carried out on a Discovery HR-1 (TA Industries) rheometer and a 40 mm plate/plate geometry according to a continuous mode (10% strain, frequency of 1 Hz, at 25° C., for 120 s). The samples consisting of around 1.2 ml are deposited in a gap of 1000 μm.

The maximum viscosity measurements are carried out in dynamic mode (angular frequency of from 0.1 to 100 rad·s⁻¹). The samples consisting of around 1.2 ml are deposited in a gap of 1000 μm.

The aqueous gel prepared according to the method of the invention may therefore be packaged in syringes in order to be able to be injected into the tissues.

A subject of the invention is thus, according to another aspect, a syringe containing the gel prepared according to the method of the invention, as described above. Such a syringe is in particular intended for filling wrinkles or fine lines.

According to this embodiment, a degassing may be carried out before filling the syringes to eliminate any possible air bubbles.

### Uses

In one particular embodiment, the aqueous gel obtained according to the present invention is intended to be used in the repair or reconstruction of tissues.

In particular, the aqueous gel according to the present invention may be used for the formation or replacement of biological tissues, for example as an implant, or the filling of biological tissues, for example injection into bone cartilage or into joints or for filling cavities of the body or face, such as wrinkles or fine lines, for creating or increasing volumes of the human body or face, or else for skin healing.

According to other particular embodiments, the aqueous gel according to the present invention may be used:
in surgery, in particular in organ repair, or in cosmetic surgery or medicine,
in urology, in particular for treating urinary incontinence,
in infectiology, in particular as carrier fluid for vaccines,
in ophthalmology, in particular for corneal healing,
in odontology, in particular for insertion of a dental implant or for bone repair,
in orthopedics, in particular in the periosteum for the creation of volume,
for cell therapy or tissue engineering, within the context of the vectorization of therapeutic cells or of dual-action factors,
   or else in angiology.

The aqueous gel according to the present invention may also be used in rheumatology.

Advantageously, the aqueous gel according to the present invention may also be used as a carrier of an active principle, in particular therapeutic active principle, such as cells, a vaccine or a hormone of insulin or estrogen type, and more generally for all active principles, the controlled and/or prolonged release or delivery of which presents an advantage.

The present invention also relates to the cosmetic use of an aqueous gel according to the invention for treating or combating skin aging.

The following example is intended to illustrate the invention without in any way limiting the scope thereof.

### Example

An aqueous hyaluronic acid gel in accordance with the invention (Composition 1) was prepared according to the following method:

Hyaluronic acid (HTL, France) was completely solubilized in an alkaline phosphate buffer solution (300 mOsmol⁻¹, pH=12.9, Merck, France) to obtain a final hyaluronic acid concentration of 150 mg·ml⁻¹.

A 20 wt % solution of BDDE (SA, France) is added slowly. The mixture is then heated at 50° C. until the texture no longer changes and the mixture is colored yellow.

The gel obtained is then rolled using an EXAKT 50i G-Line three-roll mill (Exakt, Germany). When the rolling is finished, an acid phosphate buffer solution (472 mOsmol·l⁻¹, pH=1.59) is added to neutralize the reaction mixture and to dilute the gel to a concentration of 32.5 mg·ml⁻¹ of hyaluronic acid.

The gel is then dialyzed against a phosphate buffer (300 mOsmol·l⁻¹, pH 7.4, Merck, France). The dialysis is stopped when neutrality is achieved. To finish, 4% (w/w) of a 25 mg·ml⁻¹ hyaluronic acid solution (HTL, France) are then added. The acid gel is then placed in 1 ml syringes (BD, 1 ml long) then sterilized by autoclaving (121° C. for 15 min).

A comparative aqueous hyaluronic acid gel (Composition 2) was also prepared according to the same method, apart from the rolling step which was not carried out.

The effect of the rolling on the homogeneity of the gel was demonstrated by measurement of the ejection force. The more stable the ejection force is during the expulsion of the product through the syringe and the needle, the more homogeneous the gel.

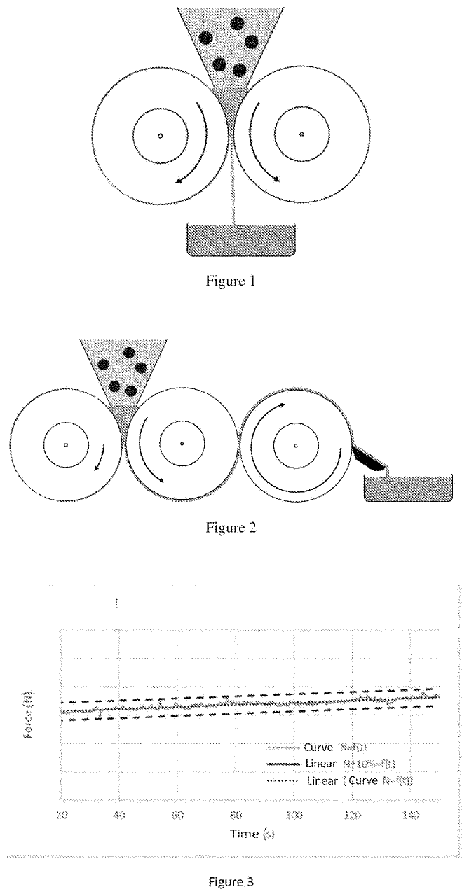

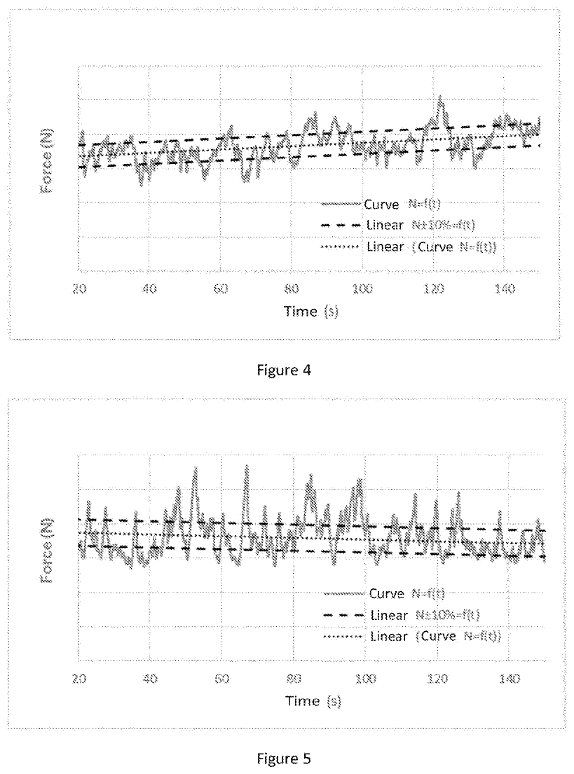

The ejection forces of compositions 1 and 2 of the aqueous gels prepared above were measured. The results of these measurements are presented in FIGS. 3 and 4.

An excellent stability of the ejection force is observed for Composition 1 according to the invention (FIG. 3). On the other hand, for Composition 2 (comparative), which is not rolled, not screened and not ground, great variations of the ejection force are observed exceeding several times the margin of ±10% relative to the linearized N=F(t) extrusion force (FIG. 4).

The ejection force was also measured for a commercial composition of aqueous crosslinked hyaluronic acid gel, Teosyal Ultradeep (Composition 3), the manufacturing method of which uses a screening/grinding step as described in patent application US 2013/0237615. The result of this measurement is presented in FIG. 5. A wide variation in the ejection force is observed during the ejection which demonstrates a heterogeneity of the gel.

1-19. (canceled)

20. A method for preparing an aqueous hyaluronic acid gel, comprising the following steps:
a) preparing an aqueous crosslinked hyaluronic acid gel;
b) homogenizing the aqueous gel formed in step (a) by rolling; and
c) neutralizing the aqueous gel homogenized in step (b).

21. The method as claimed in claim 20, wherein the rolling consists of a continuous compression between at least two counter-rotating rollers, preferably three counter-rotating rollers.

22. The method as claimed in claim 20, wherein the spacing between the counter-rotating rollers is between 20 μm and 1 mm, preferably between 20 μm and 100 μm.

23. The method as claimed in claim 20, wherein step a) of preparing an aqueous crosslinked hyaluronic acid gel comprises at least the crosslinking, in an acid or basic medium, of said hyaluronic acid in the presence of at least one crosslinking agent.

24. The method as claimed in claim 23, wherein the crosslinking, in a basic medium, of the hyaluronic acid comprises at least the following steps:
dissolving at least one hyaluronic acid and/or one of the salts thereof in a basic solution having a pH of greater than 7.5, preferably greater than or equal to 10, more preferentially between 10 and 14; and
crosslinking, in basic solution, said hyaluronic acid in the presence of at least one crosslinking agent.

25. The method as claimed in claim 23, wherein the crosslinking, in an acid medium, of the hyaluronic acid comprises at least the following steps:
dissolving at least one hyaluronic acid and/or one of the salts thereof in an acid solution having a pH of less than 6.5, preferably less than or equal to 5, more preferentially between 4.5 and 2; and
crosslinking, in acid solution, said hyaluronic acid in the presence of at least one crosslinking agent.

26. The method as claimed in claim 23, wherein the step of crosslinking the hyaluronic acid comprises at least a crosslinking, in a basic medium, of the hyaluronic acid and a crosslinking, in an acid medium, of the hyaluronic acid, and preferably a crosslinking, in a basic medium, of the hyaluronic acid followed by a crosslinking, in an acid medium, of the hyaluronic acid.

27. The method as claimed in claim 20, wherein step c) of neutralizing the hyaluronic acid solution is carried out by adjusting the pH to a pH of between 6.5 and 7.5.

28. The method as claimed in claim 20, wherein the hyaluronic acid used in the preparation of the aqueous gel in step a) has a molar mass of between 1 000 000 Da and 5 000 000 Da, preferably between 1 500 000 Da and 3 500 000 Da.

29. The method as claimed in claim 20, wherein the content of hyaluronic acid in the aqueous hyaluronic acid gel obtained in step a) is between 1 mg/ml and 300 mg/ml, preferably between 75 and 200 mg/ml, more preferentially between 100 and 175 mg/ml.

30. The method as claimed in claim 23, wherein the crosslinking agent is chosen from difunctional epoxides, multifunctional epoxides, bifunctional or polyfunctional esters, divinyl sulfones, carbodiimides, formaldehyde, dialdehydes and mixtures thereof, and preferably the crosslinking agent is 1,4-butanediol diglycidyl ether (BDDE).

31. The method as claimed in claim 23, wherein the crosslinking agent is introduced in an amount of between 10 mg and 250 mg per gram of linear hyaluronic acid introduced in step i.

32. The method as claimed in claim 24, wherein the hyaluronic acid salt is chosen from a sodium salt, a calcium salt, a zinc salt and a potassium salt, preferably a sodium salt.

33. The method as claimed in claim 20, wherein the aqueous gel is purified before or after step b) of homogenizing by rolling, said purification preferably being carried out by dialysis.

34. The method as claimed in claim 20, wherein a linear hyaluronic acid is added before or after step b) of homogenizing by rolling, the neutralization (dilution) step c) or the purification step described above.

35. The method as claimed in claim 20, wherein the aqueous gel prepared is injectable.

36. An aqueous hyaluronic acid gel capable of being obtained by the method as claimed in claim 20.

37. A method of repairing or reconstructing tissue for filling wrinkles and fine lines, comprising administering to a subject in need thereof an effective amount of the aqueous hyaluronic acid gel of claim 36.

38. An aqueous hyaluronic acid gel capable of being obtained by the method as claimed in claim 34.

29. A method for repairing or reconstructing tissues, comprising administering to a subject in need thereof an effective amount of the aqueous hyaluronic acid gel of claim 38.
